(19) United States
(12) Patent Application Publication
(10) Pub. No.: US 2016/0279346 A1
(43) Pub. Date: Sep. 29, 2016

(54) DRUG SOLUTION INJECTION NEEDLE AND TRANSDERMAL DRUG DELIVERY DEVICE

(71) Applicants: NANBU PLASTICS CO., LTD., Shizuoka (JP); THE UNIVERSITY OF TOKYO, Tokyo (JP)

(72) Inventors: Akinori INOU, Shizuoka (JP); Kotaro YOSHIMURA, Tokyo (JP); Kazuhide MINEDA, Tokyo (JP)

(73) Assignees: Nanbu Plastics Co., Ltd., Shizuoka (JP); The University of Tokyo, Tokyo (JP)

(21) Appl. No.: 15/031,653

(22) PCT Filed: Oct. 15, 2014
(86) PCT No.: PCT/JP2014/005213

§ 371 Date: Apr. 22, 2016

(30) Foreign Application Priority Data
Oct. 31, 2013 (JP) 2013-227631

(51) Int. Cl. A61M 5/32

(52) U.S. Cl. CPC A61M 5/3298; A61M 5/46

(57) ABSTRACT

A drug solution injection needle is provided that can not only inject a drug solution from multiple locations, but also restrict the depth of puncture by injection needles while preventing an observable wheal from being unlikely to form. The drug solution injection needle has multiple injection needles and a needle support for supporting the plurality of injection needles. The needle support has: a needle protruding surface from which the multiple injection needles protrude out; and an abutment fragment, which is formed in the needle protruding surface and abuts the skin to obstruct the multiple injection needles from entering the skin when the multiple injection needles have entered the skin to a predetermined depth. The abutment section is provided away from the injection needles such that an observable wheal is formed around each of the multiple injection needles.

### BACKGROUND

1. Technical Field

The present disclosure relates to a drug solution injection needle and a transdermal drug delivery device and relates especially to a drug solution injection needle having a structure that restricts the depth of puncture by an injection needle for injecting a drug solution into subcutaneous tissue and is unlikely to inhibit the formation of a wheal for confirming injection of the drug solution into the subcutaneous tissue and a transdermal drug delivery device comprising such a drug solution injection needle.

2. Description of the Related Art

A transdermal drug delivery device is used for administering a drug solution that cannot be orally administered into a human body. Devices for transdermal administration with an injection needle or devices for transdermal administration with a patch comprising a drug solution are available. However, transdermal administration with an injection needle involves pain. Further, transdermal administration with a patch comprising a drug solution takes time for the drug efficacy to be expressed and has restrictions in terms of the types of drug solutions that can be used.

The pain from transdermal administration with an injection needle is alleviated by reducing the diameter of the injection needle. However, when the diameter of an injection needle is reduced, the circulation resistance during injection of a drug solution would increase.

In this regard, a transdermal drug delivery device comprising multiple injection needles with a small diameter has been proposed in order to alleviate pain due to puncture with an injection needle and to mitigate elevation in circulation resistance.

FIG. 12 is a diagram for explaining such a conventional transdermal drug delivery device, showing the overall configuration of the transdermal drug delivery device (FIG. 12(a)), an injection needle portion and a syringe portion in a separated state (FIG. 12(b)), and the structure of the tip of the injection needle portion (FIG. 12(c)).

A transdermal drug delivery device 1 has an injection needle portion (hereinafter, referred to as a drug solution injection needle) 10 for transdermally injecting a drug solution into subcutaneous tissue and a syringe portion (hereinafter, simply referred to as a syringe) 20 for housing the drug solution and supplying the housed drug solution to the drug solution injection needle 10.

The drug solution injection needle 10 comprises multiple injection needles 15 and a needle support 11 for supporting the injection needles 15. The external shape of the needle support 11 has a cylindrical shape. In addition, on one of the end surfaces (needle protruding surface) 11a of the needle support 11, the multiple injection needles 15 are provided to protrude out from the needle protruding surface 11a. A flange 11b is formed on the other end surface of the needle support 11. Each of the injection needles 15 has a tubular structure and a discharge outlet 15a for a drug solution is formed on the tip. Further, the drug solution injection needle 10 is configured such that the depth to which the injection needles 15 pierce the patient's skin (depth of puncture) is restricted by the needle protruding surface 11a of the needle support 11. Thus, a drug solution can always be injected into a portion of subcutaneous tissue at a constant depth by injecting the drug solution into the subcutaneous tissue while the needle protruding surface 11a of the needle support 11 is in contact with the patient's skin.

The syringe 20 has a tubular body (syringe main body) 21 for housing a drug solution, a plunger 23 inserted inside the syringe main body 21, and a needle mounting section (Luer lock section) 22 formed at a tip section of the syringe main body 21. The needle mounting section 22 has an outer tubular body 22a and an inner tubular body 22b integrally formed with the syringe main body 21. The outer tubular body 22a and the inner tubular body 22b are arranged to coaxially overlap with each other. The needle mounting section 22 is configured such that the needle support 11 is mounted on the needle mounting section 22 by screwing in the flange 11b of the needle support 11 in the space between the outer tubular body 22a and the inner tubular body 22b.

Such transdermal drug delivery devices having multiple injection needles with a small diameter are disclosed in, for example, Japanese National Phase PCT Laid-open Publication No. 2005-527249, Japanese Laid-Open Publication No. 2005-87521, and Japanese Laid-Open Publication No. 2003-135598.

There is an issue, however, when such transdermal drug delivery devices are used to inject a drug solution from each injection needle to subcutaneous tissue of an affected portion, in that formation of a wheal (i.e., a swollen portion on the skin surface which is formed by injection of the drug solution into the subcutaneous tissue) for confirming injection of the drug solution into the subcutaneous tissue is inhibited.

FIG. 13 is a diagram for explaining such a problem, showing how a wheal is formed (FIG. 13(a)) and a state where wheal formation is inhibited (FIG. 13(b)).

Specifically, as shown in FIG. 13(a), when a drug solution in subcutaneously injected with a syringe S, a portion of the skin Sk where an injection needle N is pierced generally swells to form a bulging portion (wheal) Wh on the skin surface by the injection of the drug solution.

However, in the transdermal drug delivery device 1 shown in FIG. 12, in order to limit the depth of entry of the injection needles 15 into the skin to a certain depth, as shown in FIG. 13(b), a drug solution is injected while the needle protruding surface 11a of the needle support 11 is in contact with the skin Sk. That is, a portion Pc on the surface of the skin Sk in contact with the needle protruding surface 11a of the needle support 11 is pressed down upon by the needle protruding surface 11a. Thus, a wheal is unlikely to form even when a drug solution is injected into subcutaneous tissue.

This results in an issue where it is difficult to confirm the status of drug solution injection into subcutaneous tissue by a wheal.

Further, the transdermal drug delivery devices in Japanese National Phase PCT Laid-open Publication No. 2005-527249 and Japanese Laid-Open Publication No. 2005-87521 have the same issue as in the transdermal drug delivery device shown in FIG. 12. Further, the transdermal drug delivery device disclosed in Japanese Laid-Open Publication No. 2003-135598 has a structure with a recess formed on the end surface on the side of a needle support where an injection needle protrudes out (needle protruding surface). However, the device has a structure in which a drug solution is injected into subcutaneous tissue while the end surface of a depth restricting support column surrounding a microneedle abuts the skin. Thus, in the transdermal drug delivery device disclosed in Japanese Laid-Open Publication No. 2003-135598, formation of a wheal by injection of a drug solution from an injection needle is inhibited in a region surrounding an injection needle adjacent to individual injection needles.

### BRIEF SUMMARY

Embodiments of the present invention were conceived to solve the above-described problems. The objective of embodiments of the present invention is to obtain a drug solution injection needle that can not only inject a drug solution from multiple locations, but also restrict the depth of puncture by injection needles while preventing an observable wheal from being unlikely to form, and a transdermal drug delivery device comprising such a drug solution injection needle.

A drug solution injection needle according to an embodiment of the present invention has multiple injection needles and a needle support for supporting the multiple injection needles such that tip portions of the multiple injection needles protrude out is provided, where the needle support has a needle protruding surface from which the multiple injection needles protrude out, and an abutment section that abuts skin to obstruct the multiple injection needles from entering the skin when the multiple injection needles have entered the skin to a predetermined depth, the abutment section being provided on the needle protruding surface, and where the abutment section is provided away from the injection needles such that an observable wheal is formed around each of the multiple injection needles, thereby achieving the objective described above.

Preferably, in the drug solution injection needle according to an embodiment of the present invention, the abutment section is provided to avoid a space between adjacent injection needles among the multiple injection needles.

Still preferably, in the drug solution injection needle according to an embodiment of the present invention, the abutment section is arranged to surround the multiple injection needles along a circumferential edge of the needle protruding surface.

Still preferably, in the drug solution injection needle according to an embodiment of the present invention, the abutment section is discretely arranged at a predetermined interval along a circumferential edge of the needle protruding surface.

Still preferably, in the drug solution injection needle according to an embodiment of the present invention, the abutment section is arranged along a circumferential edge of the needle protruding surface so as to form a tubular shape.

Still preferably, in the drug solution injection needle according to an embodiment of the present invention, the multiple injection needles are arranged to be positioned at vertices of a polygon inside the needle protruding surface, and the abutment section is arranged in a region inside the polygon.

A transdermal drug delivery device according to an embodiment of the present invention has a syringe filled with a drug solution and a drug solution injection needle attached to a tip section of the syringe is provided, where the drug solution injection needle comprises multiple injection needles and a needle support for supporting the multiple injection needles such that tip portions of the multiple injection needles protrude out, where the needle support has a needle protruding surface from which the multiple injection needles protrude out, and an abutment section that abuts skin to obstruct the multiple injection needles from entering the skin when the multiple injection needles have entered the skin to a predetermined depth, the abutment section being provided on the needle protruding surface, and where the abutment section is provided away from the injection needles such that an observable wheal is formed around each of the multiple injection needles, thereby achieving the objective described above.

Preferably, in the transdermal drug delivery device according to an embodiment of the present invention, the abutment section is provided to avoid a space between adjacent injection needles among the multiple injection needles.

In view of the above, embodiments of the present invention can materialize a drug solution injection needle that can not only inject a drug solution from multiple locations, but also restrict the depth of puncture by injection needles while preventing an observable wheal from being unlikely to form, and a transdermal drug delivery device comprising such a drug solution injection needle.

### BRIEF DESCRIPTION OF THE SEVERAL VIEWS OF THE DRAWINGS

FIG. 1 is a diagram for schematically explaining a transdermal drug delivery device according to Embodiment 1 of the present invention, showing the overall configuration of the transdermal drug delivery device (FIG. 1(a)), the structure of an injection needle portion (cross-section at the Ib-Ib line in FIG. 1(a)) of the transdermal drug delivery device FIG. 1(b)), the structure of the tip of the injection needle portion of the transdermal drug delivery device (FIG. 1(c)), and the positional relationship between a puncture site punctured by an injection needle and an abutment region abutted by an abutment section on the skin (FIG. 1(d)).

FIG. 2 is a perspective view showing the specific structure of the injection needle portion of the transdermal drug delivery device according to Embodiment 1 of the present invention.

FIG. 3 is a diagram for explaining a method of assembling the transdermal drug delivery device according to Embodiment 1 of the present invention, FIG. 3 showing the procedure (FIGS. 3(a)-3(d)) for attaching the injection needle portion housed in a needle holder to a syringe.

FIG. 4 is a diagram for schematically explaining a transdermal drug delivery device according to Embodiment 2 of the present invention, showing the overall configuration of the transdermal drug delivery device (FIG. 4(a)), the structure of an injection needle portion (cross-section at the IVb-IVb line in FIG. 4(a)) of the transdermal drug delivery device (FIG. 4(b)), the structure of the tip of the injection needle portion of the transdermal drug delivery device (FIG. 4(c)), and the positional relationship between a puncture site punctured by an injection needle and an abutment region abutted by an abutment section on the skin (FIG. 4(d)).

FIG. 5 is a perspective view showing the specific structure of the injection needle portion of the transdermal drug delivery device according to Embodiment 2 of the present invention.

FIG. 6 is a diagram for schematically explaining a transdermal drug delivery device according to Embodiment 3 of the present invention, showing the overall configuration of the transdermal drug delivery device (FIG. 6(a)), the structure of an injection needle portion (cross-section at the VIb-VIb line in FIG. 6(a)) of the transdermal drug delivery device (FIG. 6(b)), the structure of the tip of the injection needle portion of the transdermal drug delivery device (FIG. 6(c)), and the positional relationship between a puncture site punctured by an injection needle and an abutment region abutted by an abutment section on the skin (FIG. 6(d)).

FIG. 7 is a perspective view showing the specific structure of the injection needle portion of the transdermal drug delivery device according to Embodiment 3 of the present invention.

FIG. 8 is a diagram for schematically explaining a transdermal drug delivery device according to Embodiment 4 of the present invention, showing the overall configuration of the transdermal drug delivery device (FIG. 8(a)), the structure of an injection needle portion (cross-section at the VIIIb-VIIIb line in FIG. 8(a)) of the transdermal drug delivery device (FIG. 8(b)), the structure of the tip of the injection needle portion of the transdermal drug delivery device (FIG. 8(c)), and the positional relationship between a puncture site punctured by an injection needle and an abutment region abutted by an abutment section on the skin (FIG. 8(d)).

FIG. 9 is a perspective view showing the specific structure of the injection needle portion of the transdermal drug delivery device according to Embodiment 4 of the present invention.

FIG. 10 is a diagram for schematically explaining a transdermal drug delivery device according to Embodiment 5 of the present invention, showing the overall configuration of the transdermal drug delivery device (FIG. 10(a)), the structure of an injection needle portion (cross-section at the Xb-Xb line in FIG. 10(a)) of the transdermal drug delivery device (FIG. 10(b)), the structure of the tip of the injection needle portion of the transdermal drug delivery device (FIG. 10(c)), and the positional relationship between a puncture site punctured by an injection needle and an abutment region abutted by an abutment section on the skin (FIG. 10(d)).

FIG. 11 is a perspective view showing the specific structure of the injection needle portion of the transdermal drug delivery device according to Embodiment 5 of the present invention.

FIG. 12 is a diagram for explaining a conventional transdermal drug delivery device, showing the overall configuration of the transdermal drug delivery device (FIG. 12(a)), separated state of an injection needle portion and a syringe (FIG. 12(b)), and the structure of the injection needle portion (FIG. 12(c)).

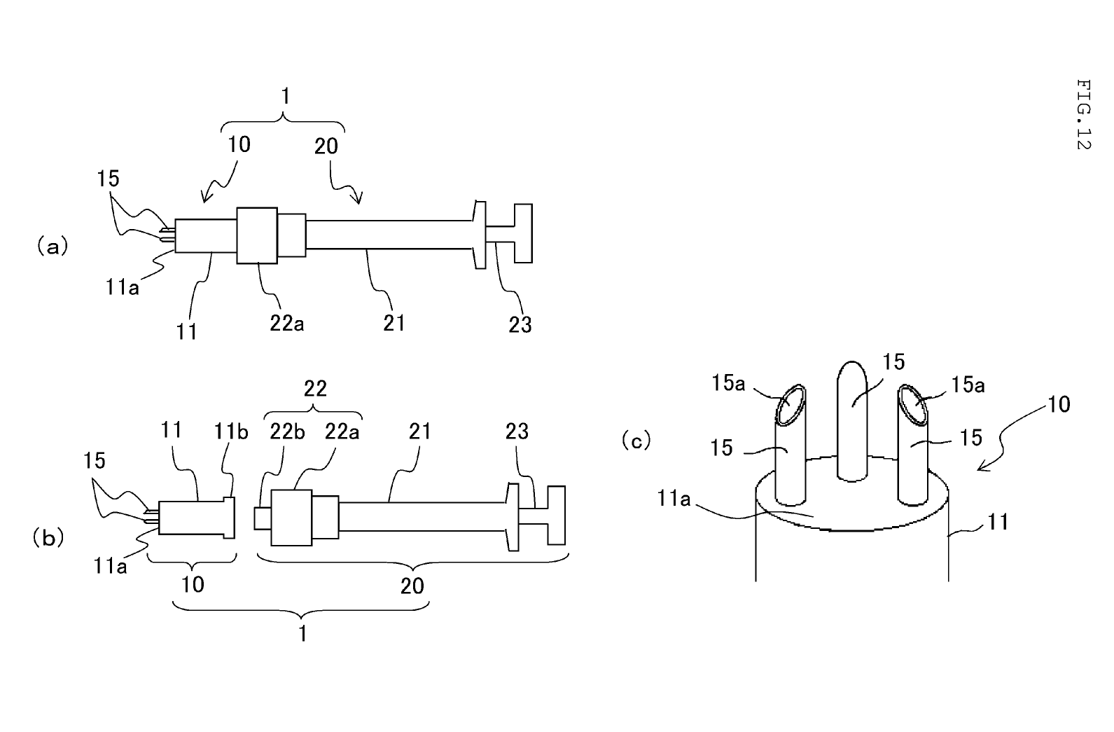

FIG. 13 is a diagram for explaining the problems in a conventional transdermal drug delivery device, showing how a wheal is formed (FIG. 13(a)) and a state where wheal formation is inhibited (FIG. 13(b)).

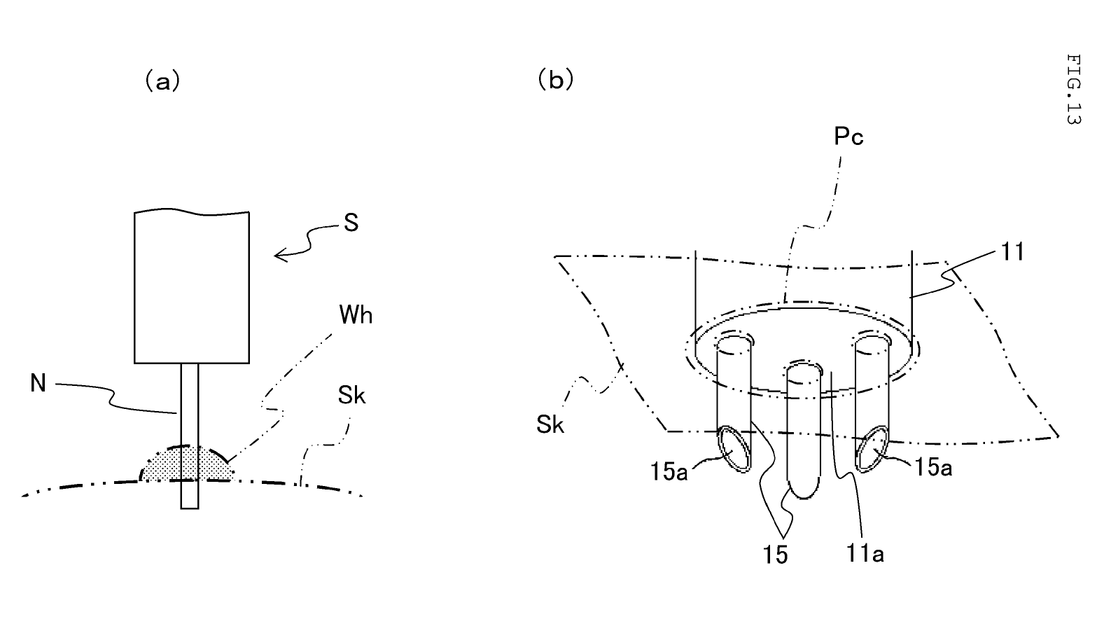

### DETAILED DESCRIPTION

The embodiments of the present invention are explained hereinafter while referring to the drawings.

### Embodiment 1

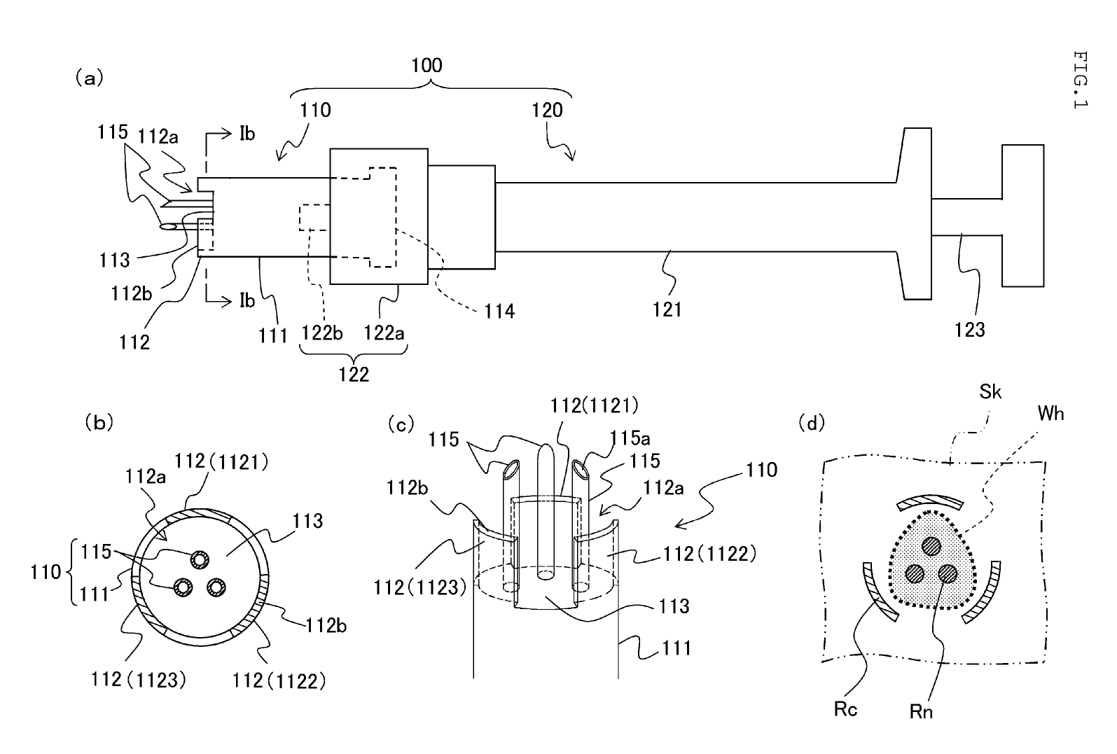

FIG. 1 is a diagram for schematically explaining a transdermal drug delivery device according to Embodiment 1 of the present invention, showing the overall configuration of the transdermal drug delivery device (FIG. 1(a)), the structure of an injection needle portion (cross-section at the Ib-Ib line in FIG. 1(a)) of the transdermal drug delivery device (FIG. 1(b)), the structure of the tip of the injection needle portion of the transdermal drug delivery device (FIG. 1(c)), and the positional relationship between a puncture site punctured by an injection needle and an abutment region abutted by an abutment section on the skin (FIG. 1(d)).

A transdermal drug delivery device 100 according to Embodiment 1 has an injection needle portion (hereinafter, referred to as a drug solution injection needle) 110 for transdermally injecting a drug solution into subcutaneous tissue and a syringe portion (hereinafter, simply referred to as a syringe) 120 for housing the drug solution and supplying the housed drug solution to the drug solution injection needle 110. The drug solution injection needle 110 is attached to a tip section of the syringe 120.

The drug solution injection needle 110 comprises multiple injection needles 115 and a needle support 111 for supporting the multiple injection needles 115. The needle support 111 has a needle protruding surface 113 from which the multiple injection needles 115 are protruding out and an abutment section 112 formed on the needle protruding surface 113. The external shape of the needle support 111 has a roughly cylindrical shape. In addition, on one of the end surfaces (needle protruding surface) 113, the multiple injection needles 115 are provided to protrude out from the needle protruding surface 113. A flange 114 is formed on the other end surface of the needle support 111. Each of the injection needles 115 has a tubular structure and a discharge outlet 115a for a drug solution is formed on the tip.

The abutment section 112 is a portion that abuts the skin to obstruct the multiple injection needles 115 from entering the skin when the multiple injection needles 115 have entered the skin to a predetermined depth. Herein, the abutment section 112 is provided away from the injection needles 115 such that an observable wheal is formed around each of the multiple injection needles 115 as shown in FIG. 1(b). Furthermore, the abutment section 112 is arranged to avoid the space between adjacent injection needles 115 among the multiple injection needles 115. For example, the abutment section 112 and each of the injection needles 115 are arranged herein with a space of, for example, about 1 mm to 10 mm such that a verifiable wheal is formed between the abutment section 112 and the injection needles 115. The end surface on the needle tip side of the abutment section 112 is a skin abutting surface 112b that abuts the skin. Thus, an abutment region Rc on skin Sk to which the abutment section 112 abuts, as shown in FIG. 1(d), is positioned sufficiently away from a puncture site Rn on the skin where the injection needle 115 pierces for a wheal Wh to form between the puncture site Rn and the abutment region Rc. Further, the abutment region Rc is not present at least between two adjacent puncture sites Rn among multiple puncture sites Rn on the skin Sk where the multiple injection needles 115 pierce.

Further, the syringe 120 has a tubular body (syringe main body) 121 for housing a drug solution, a plunger 123 inserted inside the syringe main body 121, and a needle mounting section (Luer lock section) 122 formed at a tip section of the syringe main body 121. The needle mounting section 122 has an outer tubular body 122a and an inner tubular body 122b integrally formed with the syringe main body 121 and the outer tubular body 122a and the inner tubular body 122b are arranged to coaxially overlap with each other. The needle mounting section 122 is configured such that the drug solution injection needle 110 is mounted onto the syringe 120 by screwing in the flange 114 of the needle support 111 in the space between the outer tubular body 122a and the inner tubular body 122b.

The drug solution injection needle 110 is explained in detail hereinafter.

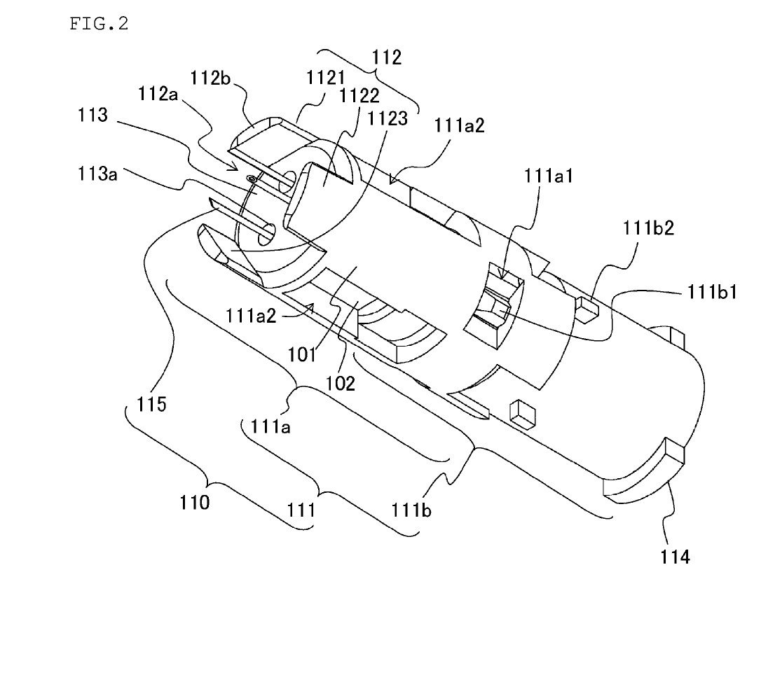

FIG. 2 is a perspective view showing the specific structure of the drug solution injection needle (injection needle portion) 110 according to Embodiment 1 of the present invention.

The needle support 111 of the drug solution injection needle 110 has a needle support main body 111b to which three injection needles 115 are attached and a needle support cap 111a for restricting the depth of puncture of the injection needles 115 into the skin, which is attached to one of the end sections of the needle support main body 111b.

Herein, a foundation portion of the injection needles 115 is embedded into one of the end sections of the needle support main body 111b. The flange 114 for mounting the drug solution injection needle 110 onto the syringe 120 is formed on the other end section of the needle support main body 111b.

Further, the needle support cap 111a is attached to the needles support main body 111b by covering the cylindrical body constituting the needle support main body 111b with a cylindrical body constituting the needle support cap 111a and engaging an engagement protrusion 111b1 formed on the outer circumferential surface of the cylindrical body of the needle support main body 111b with an engagement bore 111a1 formed on a side wall of the cylindrical body of the needle support cap 111a. Needle through holes 113a allowing the injection needles 115 to protrude out are formed on the surface on the tip side of the injection needles 115 on the needle support cap 111a. The injection needles 115 protrude out from the through holes 113a. That is, the surface on the tip side of the injection needles 115 on the needle support cap 111a is the needle protruding surface 113 of the needle support 111. A rotation restricting protrusion 111b2 for engaging a groove in a needle holder so as to restrict the rotation of the drug solution injection needle 110 in the needle holder for housing the drug solution injection needle 110 is formed on the outer circumferential surface of the cylindrical body constituting the needle support body 111b.

Three sheet-shaped abutment fragments 1121-1123 used as the abutment section 112 are arranged at equidistance along the circumferential edge of the needle protruding surface 113 to surround the three injection needles 115 on the needle protruding surface 113. Each of the sheet-shaped abutment fragments 1121-1123 is arranged to oppose one of the three injection needles 115. Needless to say, the opposing injection needles 115 and the sheet-shaped abutment fragments 1121-1123 are arranged herein with a space (e.g., 1 mm to 10 mm) sufficient for an observable wheal to form therebetween. Herein, the height of the sheet-shaped abutment fragments 1121-1123 is, for example, 1 mm to 10 mm. The thickness of the sheet-like abutment fragments 1121-1123 is, for example, 0.5 mm to 2.0 mm.

Furthermore, the length of the injection needles 115 protruding out from the needle protruding surface 113 is longer than the height of the sheet-like abutment fragments 1121-1123 on the needle protruding surface 113. A space corresponding to the height of the sheet-like abutment fragments 1121-1123, i.e., space from the needle protruding surface 113 to the skin abutment surface 112b of each of sheet-like abutment fragments 1121-1123, is a wheal formation space 112a for ensuring that formation of a wheal (swelling on the skin) by injection of a drug solution is not inhibited.

Further, the needle support cap 111a of the needle support 111 comprises an outer shell section 101 comprising the needle protruding surface 113 and the sheet-like abutment fragments 1121-1123 and a needle guiding member 102 integrated with the outer shell section 101, for guiding the injection needles 115 attached to the needle support main body 111b to the needle through hole 113a of the needle protruding surface 113. A positioning aperture 111a2 for positioning a needling guiding channel (not shown) of the needle guiding member 102 and the needle through hole 113a of the needle protruding surface 113 of the needle support cap 111a is formed on the outer shell section 101 of the needle support cap 111a.

The needle support main body 111b of the needle support 111 and the needle guiding member 102 and the outer shell section 101 constituting the needle support cap 111a of the needle support 111 can be manufactured by injection molding or the like with a thermoplastic resin such as polycarbonate, polypropylene, ABS resin, or polystyrene as the material. Examples of constituent material for the injection needles 115 include, but are not limited to, metallic materials such as stainless steel, aluminum or aluminum alloy, and titanium or titanium alloy.

Further, the three injection needles 115 are arranged equidistant from the center of the circular needle protruding surface 113, and the inscribed angle formed by adjacent lines, among the three lines connecting each of the three injection needles 115 and the center of the needle protruding surface 113, is 120°.

For example, when the diameter of the needle protruding surface 113 of the drug solution injection needle 110 is 5 to 20 mm and the three injection needles 115 are provided on the needle protruding surface 113, the distance of each of the injection needles 115 from the center of the needle protruding surface 113 is preferably 1 to 5 mm and more preferably 1 to 1.5 mm.

Further, the pitch (distance) between adjacent injection needles 115 is preferably 1 to 10 mm, more preferably 1 to 5 mm, and most preferably 1 to 3 mm.

The dimension of the injection needles 115 protruding out from the skin abutment surface 112b of the sheet-like abutment section 112 is preferably 0.1 to 3 mm or less, more preferably about 0.5 to 2.0 mm, and still more preferably about 0.5 to 1.5 mm. The maximum outer diameter of the injection needles 115 is preferably about 0.1 to 0.6 mm, and more preferably about 0.2 to 0.6 mm. The tip of the injection needles 115 has a shape, which would be formed by diagonally cutting a tubular member.

However, the number, arrangement, or shape of the injection needles 115 at the needle support 111 is not limited to those described above. Any needle support 111 can be used which can spread the locations where a drug solution is injected into the skin by the multiple injection needles 115.

Furthermore, a drug solution used in the transdermal drug delivery device 100 is typically a solution, a gel, or a suspension containing an agent. Agents that can be used are not substantially limited, as long as it is not an agent that is unsuitable for transdermal administration.

Examples of preferred agents transdermally administered with the transdermal drug delivery device 100 include proteins, peptides, polysaccharides, oligonucleotides, DNA and the like whose effect is not expressed or is diminished by oral administration. Specific examples thereof include high molecular weight medicaments such as insulin, growth hormones, interferon and calcitonin.

The next section explains a method of assembling the transdermal drug delivery device 100 by attaching the injection needle portion (drug solution injection needle) 110 to the syringe 120.

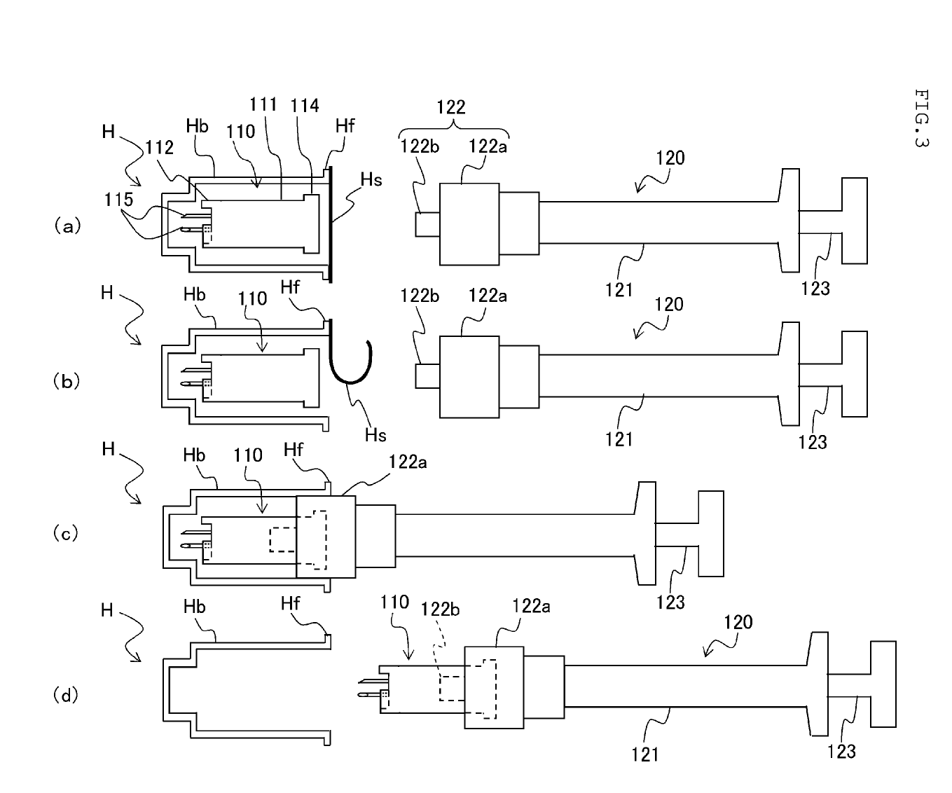

FIG. 3 is a diagram for explaining a method of assembling the transdermal drug delivery device according to Embodiment 1 of the present invention. FIGS. 3(a)-3(d) show the procedure for attaching the injection needle portion (drug solution injection needle) housed in a needle housing container (needle holder) to a syringe.

The drug solution injection needle 110 is provided while being housed in a sealed needle holder H. The needle holder H has a holder main body Hb for housing the drug solution injection needle 110, a holder flange Hf formed along the opening of the holder main body Hb, and a sealing lid member Hs applied to the holder flange Hf. The drug solution injection needle 110 is housed in the holder main body Hb while the rotation restricting protrusion 111b2 formed on the outer surface of the needle support main body 111b of the needle support 111 is engaged with a groove (not shown) in the holder main body Hb such that the drug solution injection needle 110 does not rotate within the needle holder H. In addition, the drug solution injection needle is sealed aseptically within the holder main body Hb with the sealing lid member Hs applied to the holder flange Hf. When a drug solution is administered to individual patients, the needle holder H is unsealed and the drug solution injection needle 110 is mounted onto the syringe 120.

Specifically, the needle holder H housing a new drug solution injection needle 110 and the syringe 120 are first prepared (FIG. 3(a)), and the sealing lid member Hs of the needle holder H is peeled off (FIG. 3(b)). The mounting section 122 of the syringe 120 is screwed into the end section of the drug solution injection needle 110 such that the flange 114 of the drug solution injection needle 110 housed in the needle holder H is inserted between the outer tubular body 122a and the inner tubular body 122b of the mounting section 122 of the syringe 120 (FIG. 3(c)). The drug solution injection needle 110 is then taken out of the needle holder H while the drug solution injection needle 110 is mounted onto the syringe 120 (FIG. 3(d)). The transdermal drug delivery device 100 in which the drug solution injection needle 110 is mounted onto the tip of the syringe 120 is thereby assembled.

The syringe 120 of the transdermal drug delivery device 100 is then filled with a drug solution, and the transdermal drug delivery device 100 is used to transdermally administered the drug solution.

Specifically, the injection needle 115 is inserted into the skin, and while the skin abutment surface 112b of the abutment section 112 (sheet-like abutment fragments 1121-1123) of the drug solution injection needle 110 is abutting the patient's skin, the plunger 123 is pushed in to inject the drug solution into subcutaneous tissue of a patient.

At this time, the positional relationship between the puncture site Rn punctured by the injection needle 115 and the region (abutment region) Rc where the skin abutment surface 112b of the abutment section 112 contacts the skin surface is as shown in FIG. 1(d). That is, the abutment region Rc is positioned with a space (1 mm to 10 mm) sufficient or more than sufficient for the observable wheal Wh to form between the abutment region Rc and the puncture site Rn. Further, there is no abutment region Rc between puncture sites Rn of adjacent injection needles 115.

In this manner, Embodiment 1 has the needle support 111 for supporting multiple injection needles 115 such that the tip of each injection needle protrudes out in the drug solution injection needle 110 for transdermal administration of a drug solution. Thus, a drug solution can be injected to more uniformly spread the drug solution in skin tissue by spreading out the injection locations of the drug solution.

Further, as the abutment section 112 for obstructing multiple injection needles from entering the skin by abutting the skin when the multiple injection needles have entered the skin to a predetermined depth, the sheet-like abutment fragments 1121-1123 are provided with a space (e.g., 1 mm to 10 mm) sufficient for an observable wheal to form between the sheet-like abutment fragments and the injection needles 115. Thus, a wheal indicating that a drug solution has been injected can be observed when the drug solution is injected into subcutaneous tissue by using the transdermal drug delivery device 100 of Embodiment 1.

Further, multiple sheet-like abutment fragments 1121-1123 are arranged at equidistance to surround the multiple injection needles 115. Thus, the sheet-like abutment fragments 1121-1123 would not be present between adjacent injection needles 115, thereby preventing a wheal from being unlikely to form between adjacent injection needles 115 due to the sheet-like abutment fragments 1121-1123 for restricting the depth of puncture by an injection needle.

This results in embodiments of the present invention being able to realize the drug solution injection needle 110 that can not only inject a drug solution from multiple locations, but also restrict the depth of puncture by injection needles while preventing an observable wheal from being unlikely to form, and the transdermal drug delivery device 100 comprising the drug solution injection needle 110.

### Embodiment 2

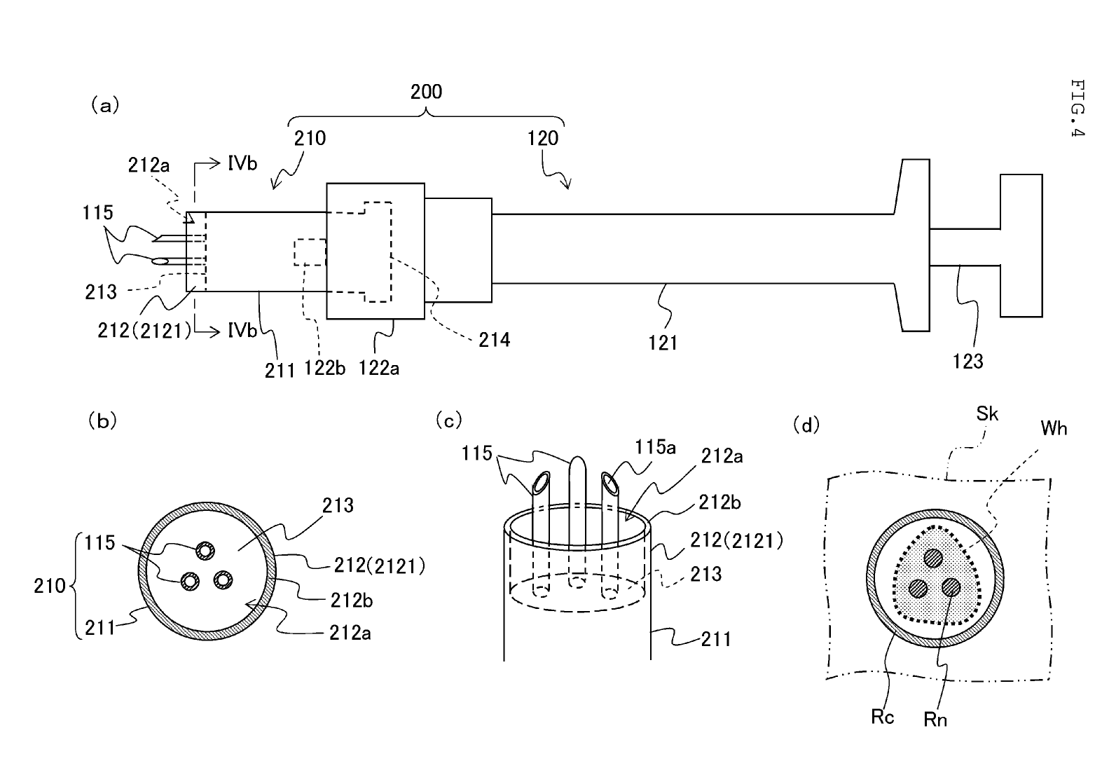

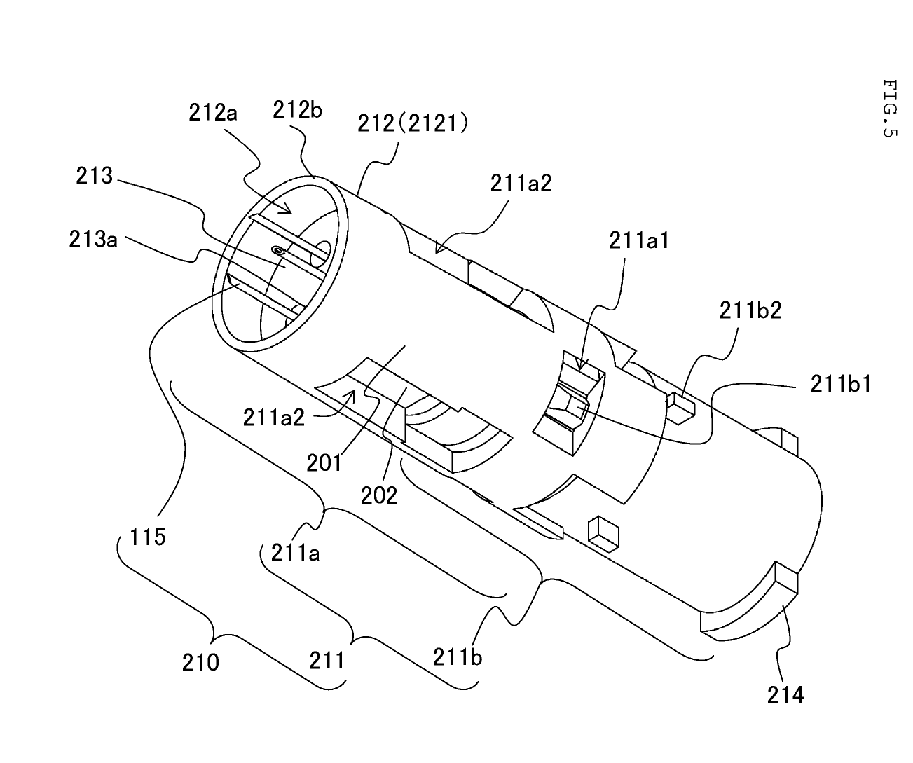

FIG. 4 is a diagram for schematically explaining a transdermal drug delivery device according to Embodiment 2 of the present invention, showing the overall configuration of the transdermal drug delivery device (FIG. 4(a)), the structure of an injection needle portion (cross-section at the IVb-IVb line in FIG. 4(a)) of the transdermal drug delivery device (FIG. 4(b)), the structure of the tip of the injection needle portion of the transdermal drug delivery device (FIG. 4(c)), and positional relationship of a puncture site punctured by the injection needle and an abutment region abutted by an abutment section on the skin (FIG. 4(d)). FIG. 5 is a perspective view showing the specific structure of the injection needle portion of the transdermal drug delivery device according to Embodiment 2 of the present invention.

A transdermal drug delivery device 200 according to Embodiment 2 comprises, instead of the drug solution injection needle 110 in the transdermal drug delivery device 100 according to Embodiment 1, a drug solution injection needle 210 with a structure that is different from the drug solution injection needle 110.

Specifically, the drug solution injection needle 210 has, as in the drug solution injection needle 110 according to Embodiment 1, multiple (three) injection needles 115 and a needle support 211 for supporting the multiple injection needles 115. The needle support 211 has a circular needle protruding surface 213 from which the multiple injection needles 115 protrude out and an abutment section 212 that abuts the skin to obstruct the multiple injection needles 115 from entering the skin when the multiple injection needles 115 have entered the skin to a predetermined depth.

Herein, the abutment section 212, unlike the sheet-like abutment fragments 1121-1123 arranged equally along the circumferential edge of the needle protruding surface 113 in Embodiment 1, is structured to be arranged along the circumferential edge of the circular needle protruding surface 213 so as to form a cylindrical shape. That is, the abutment section 212 is comprised of a cylindrical abutment fragment 2121 having the same diameter as the circular needle protruding surface 213. Thus, the cylindrical abutment fragment 2121 used as the abutment section 212 is positioned away from the injection needles 115 and positioned to avoid the space between adjacent injection needles 115 among the multiple injection needles 115, as shown in FIG. 4(b), such that an observable wheal is formed around each of the multiple injection needles 115.

For instance, the cylindrical abutment fragment 2121 is provided with a space (e.g., 1 mm to 10 mm) sufficient for an observable wheal to form between the cylindrical abutment fragment 2121 and the injection needles 115. The height of the cylindrical abutment fragment 2121 is, for example, 1 mm to 10 mm, and the thickness of the cylindrical abutment fragment 2121 is, for example, 0.5 mm to 2.0 mm. Herein, a space corresponding to the height of the cylindrical abutment fragment 2121, i.e., space from the needle protruding surface 213 to a skin abutment surface 212b of the cylindrical abutment fragment 2121, is a wheal formation space 212a for ensuring that swelling of the skin by injection of a drug solution is not inhibited.

A needle support cap 211a, needle support main body 211b, outer shell section 201, needle guiding member 202, needle through hole 213a, flange 214, engagement bore 211a1, engagement protrusion 211b1, aperture 211a2, and rotation restricting protrusion 211b2 in the needle support 211 of the transdermal drug delivery device 200 according to Embodiment 2 are identical to the needle support cap 111a, needle support main body 111b, outer shell section 101, needle guiding member 102, needle through hole 113a, flange 114, engagement bore Mal, engagement protrusion 111b1, aperture 111a2, and rotation restricting protrusion 111b2 in Embodiment 1, respectively.

Also in Embodiment 2, the plunger 123 is pushed in to inject a drug solution into subcutaneous tissue of a patient while the injection needle 115 is inserted into the skin until the cylindrical abutment fragment 1121 of the transdermal drug delivery device 200 abuts the skin. While in such a state where the injection needle 115 is inserted into the skin, the positional relationship between a puncture site Rn punctured by the injection needle 115 and an abutment region Rc where the skin abutment surface 212b of the abutment section 212 abuts the skin surface is as shown in FIG. 4(d).

That is, the abutment region Rc and the puncture site Rn are positioned with a space sufficient or more than sufficient for an observable wheal Wh to form therebetween. The abutment region Rc is not present between the puncture sites Rn of adjacent injection needles 115.

Embodiment 2 with such a configuration has the needle support 211 for supporting the multiple injection needles 115 such that the tip of each injection needle protrudes out from the drug solution injection needle 210 for transdermal administration of a drug solution. Thus, a drug solution can be injected to uniformly spread the drug solution in skin tissue by spreading out the locations of drug solution injection.

Further, as the abutment section 212 for obstructing the multiple injection needles 115 from entering the skin by abutting the skin when the multiple injection needles 115 have entered the skin to a predetermined depth, the cylindrical abutment fragment 2121 is provided to surround the multiple injection needles 115 with a space (e.g., 1 mm to 10 mmm) sufficient for an observable wheal to form between the cylindrical abutment fragment 2121 and the injection needles 115. Thus, when the transdermal drug delivery device 200 according to Embodiment 2 is used to inject a drug solution into subcutaneous tissue, a wheal indicating that the drug solution has been injected can be observed.

Further, the cylindrical abutment fragment 2121 is arranged to surround the multiple injection needles 115. Thus, the cylindrical abutment fragment 2121 would not be present between adjacent injection needles 115, thereby preventing a wheal from being unlikely to form between adjacent injection needles 115 due to the cylindrical abutment fragment 2121 for restricting the depth of puncture by an injection needle.

This results in embodiments of the present invention being able to realize the drug solution injection needle 210 that can not only inject a drug solution from multiple locations, but also restrict the depth of puncture by injection needles while preventing an observable wheal from being unlikely to form, and the transdermal drug delivery device 200 comprising the drug solution injection needle 210.

### Embodiment 3

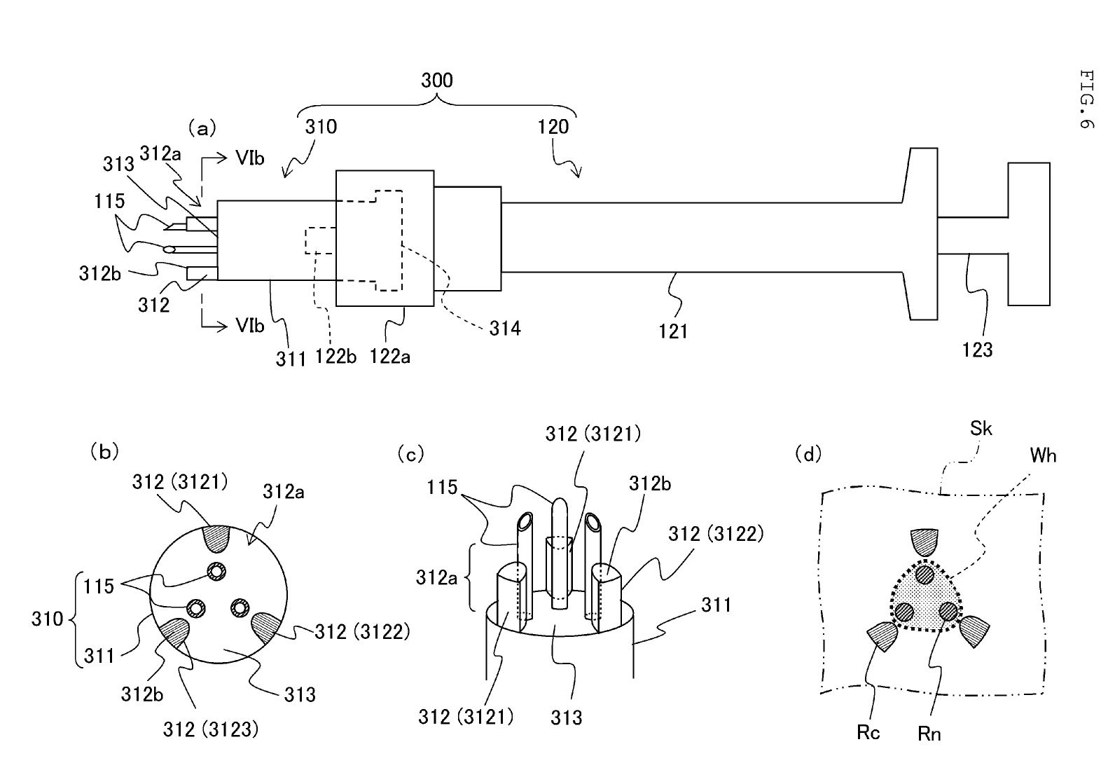

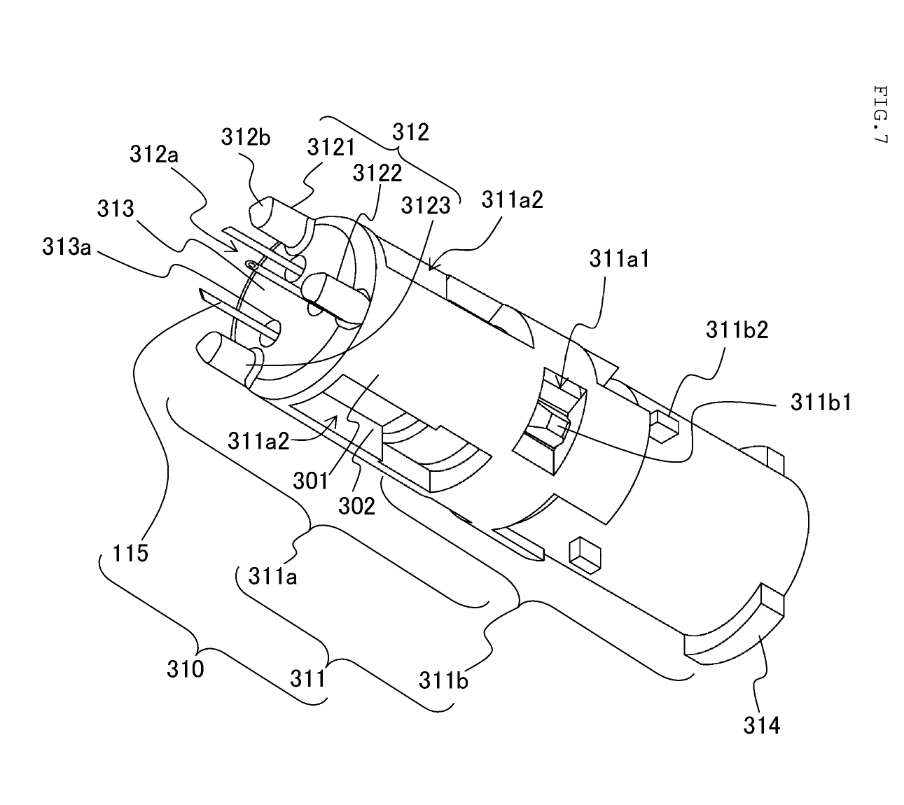

FIG. 6 is a diagram for schematically explaining a transdermal drug delivery device according to Embodiment 3 of the present invention, showing the overall configuration of the transdermal drug delivery device (FIG. 6(a)), the structure of an injection needle portion (cross-section at the VIb-VIb line in FIG. 6(a)) of the transdermal drug delivery device (FIG. 6(b)), the structure of the tip of the injection needle portion of the transdermal drug delivery device (FIG. 6(c)), and the positional relationship between a puncture site punctured by the injection needle and an abutment region abutted by an abutment section on the skin (FIG. 6(d)). FIG. 7 is a perspective view showing the specific structure of the injection needle portion of the transdermal drug delivery device according to Embodiment 3 of the present invention.

A transdermal drug delivery device 300 according to Embodiment 3 comprises a drug solution injection needle 310 with a structure that is different from the drug solution injection needle 110 according to Embodiment 1 and the drug solution injection needle 210 according to Embodiment 2.

Specifically, the drug solution injection needle 310 has, as in the drug solution injection needle 110 according to Embodiment 1 or the drug solution injection needle 210 according to Embodiment 2, multiple (three) injection needles 115 and a needle support 311 for supporting the injection needles 115. The needle support 311 has a needle protruding surface 313 from which the multiple injection needles 115 protrude out and an abutment section 312 that abuts the skin to obstruct the multiple injection needles 115 from entering the skin when the multiple injection needles 115 have entered the skin to a predetermined depth.

Herein, the needle support 311 comprises, instead of the sheet-like abutment fragments 1121-1123 in the needle support 111 of the drug solution injection needle 110 according to Embodiment 1, columnar abutments 3121-3123 as the abutment section 312. The columnar abutments 3121-3123 are arranged at equidistance along the circumferential edge of the circular needle protruding surface 313. Specifically, the columnar abutments 3121-3123 used as the abutment section 312 are arranged away from the injection needles 115 and arranged to avoid the space between adjacent injection needles 115 among the multiple injection needles 115, as shown in FIG. 6(b), such that an observable wheal is formed around each of the multiple injection needles 115.

For instance, opposing injection needles 115 and the sheet-like abutments 3121-3123 are arranged with a space (e.g., 1 mm to 10 mm) sufficient for an observable wheal to form therebetween. Further, the height of the columnar abutments 3121-3123 is, for example, 1 mm to 10 mm, and each of the dimension in the radial direction and the thickness in the direction orthogonal thereto of the columnar abutments 3121-3123 is, for example, 0.5 mm to 2.0 mm. The cross-sectional shape of the columnar abutments 3121-3123 is a bullet-like shape (shape near the apex of a graph of a quadratic equation). Further, a space corresponding to the height of the columnar abutments 3121-3123, i.e., space from the needle protruding surface 313 to the skin abutment surface 312b of the columnar abutments 3121-3123 is a wheal formation space 312a for ensuring that swelling on the skin by injection of a drug solution is not inhibited.

A needle support cap 311a, needle support main body 311b, outer shell section 301, needle guiding member 302, needle through hole 313a, flange 314, engagement bore 311a1, engagement protrusion 311b1, aperture 311a2, and rotation restricting protrusion 311b2 in the needle support 311 of the transdermal drug delivery device 300 according to Embodiment 3 are identical to the needle support cap 111a, needle support main body 111b, outer shell section 101, needle guiding member 102, needle through hole 113a, flange 114, engagement bore Mal, engagement protrusion 111b1, aperture 111a2, and rotation restricting protrusion 111b2 in Embodiment 1, respectively.

Also in Embodiment 3, the plunger 123 is pushed in to inject a drug solution into subcutaneous tissue of a patient while the injection needle 115 is inserted into the skin until the columnar abutments 3121-3123 of the transdermal drug delivery device 300 abut the skin. While in such a state where the injection needle 115 is inserted into the skin, the positional relationship between a puncture site Rn punctured by the injection needle 115 and an abutment region Rc where the skin abutment surface 312b of the abutment section 312 abuts the skin surface is as shown in FIG. 6(d).

That is, the abutment region Rc and the puncture site Rn are positioned with a space sufficient or more than sufficient for an observable wheal Wh to form therebetween. The abutment region Rc is not present between the puncture sites Rn of adjacent injection needles 115.

Embodiment 3 with such a configuration has the needle support 311 for supporting the multiple injection needles 115 such that the tip of each injection needle protrudes out in the drug solution injection needle 310 for transdermal administration of a drug solution. Thus, a drug solution can be injected to uniformly spread the drug solution in skin tissue by spreading out the locations of drug solution injection.

Further, as the abutment section 312 for obstructing the multiple injection needles 115 from entering the skin by abutting the skin when the multiple injection needles 115 have entered the skin to a predetermine depth, the columnar abutments 3121-3123 are provided with a space (e.g., 1 mm to 10 mmm) sufficient for an observable wheal to form between the columnar abutments 3121-3123 and the injection needles 115. Thus, when the transdermal drug delivery device 300 according to Embodiment 3 is used to inject a drug solution into subcutaneous tissue, a wheal indicating that the drug solution has been injected can be observed.

Further, the columnar abutments 3121-3123 are arranged to oppose each of the injection needles 115 while avoiding the space between adjacent injection needles 115 among the multiple injection needles 115. Thus, the columnar abutments 3121-3123 would not be present between adjacent injection needles 115, thereby preventing a wheal from being unlikely to form due to the columnar abutments 3121-3123 for restricting the depth of puncture by an injection needle.

This results in embodiments of the present invention being able to realize the drug solution injection needle 310 that can not only inject a drug solution from multiple locations, but also restrict the depth of puncture by injection needles while preventing an observable wheal from being unlikely to form, and the transdermal drug delivery device 300 comprising the drug solution injection needle 310.

### Embodiment 4

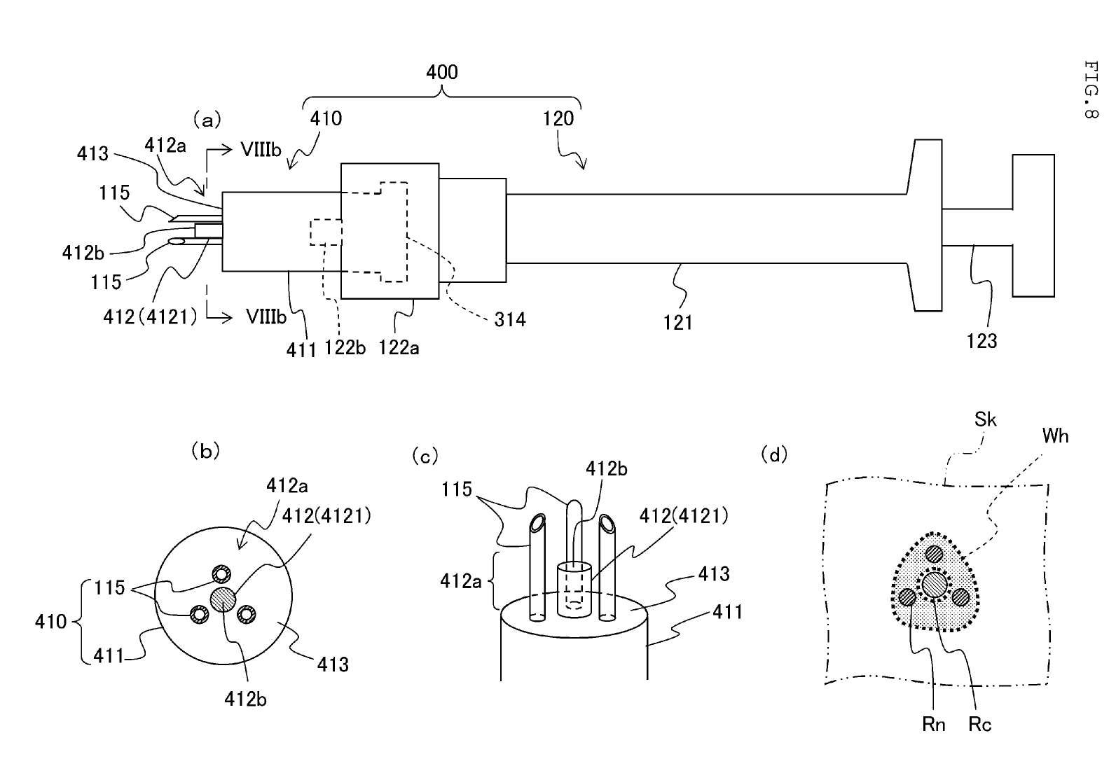

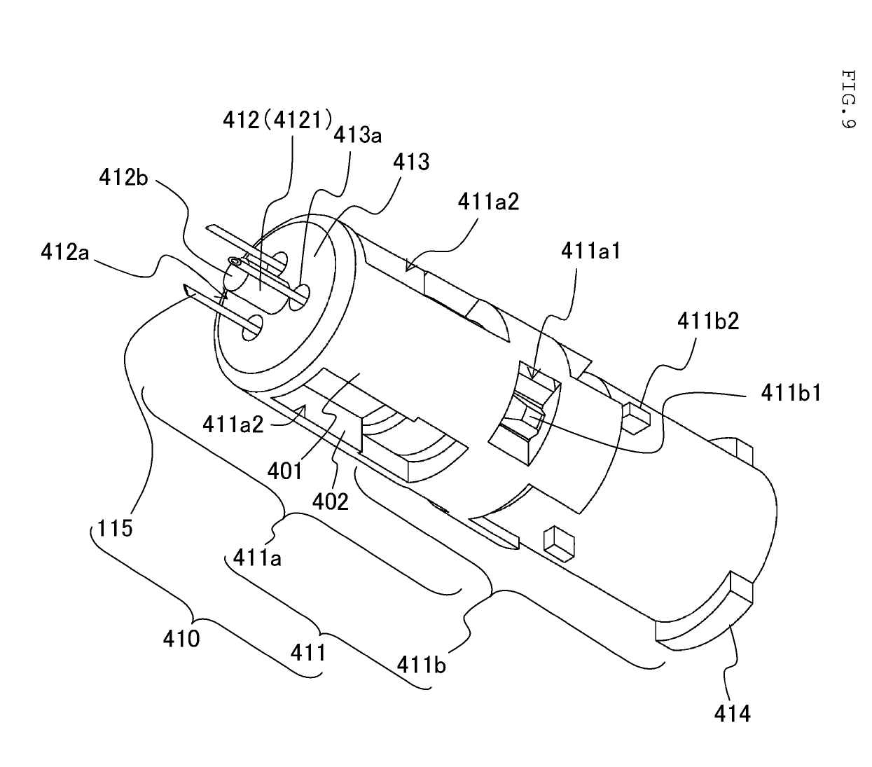

FIG. 8 a diagram for schematically explaining a transdermal drug delivery device according to Embodiment 4 of the present invention, showing the overall configuration of the transdermal drug delivery device (FIG. 8(a)), the structure of an injection needle portion (cross-section at the VIIIb-VIIIb line in FIG. 8(a)) of the transdermal drug delivery device (FIG. 8(b)), the structure of the tip of the injection needle portion of the transdermal drug delivery device (FIG. 8(c)), and the positional relationship of a puncture site punctured by the injection needle and an abutment region abutted by an abutment section on the skin (FIG. 8(d)). FIG. 9 is a perspective view showing the specific structure of the injection needle portion of the transdermal drug delivery device according to Embodiment 4 of the present invention.

A transdermal drug delivery device 400 according to Embodiment 4 comprises a drug solution injection needle 410 with a structure that is different from the drug solution injection needle 110 according to Embodiment 1, the drug solution injection needle 210 according to Embodiment 2, and the drug solution injection needle 310 according to Embodiment 3.

Specifically, the drug solution injection needle 410 has, as in the drug solution injection needles 110, 210, and 310 according to Embodiments 1, 2, and 3, multiple (three) injection needles 115 and a needle support 411 for supporting the injection needles 115. The needle support 411 has a circular needle protruding surface 413 from which the multiple injection needles 115 protrude out and an abutment section 412 that abuts the skin to obstruct the multiple injection needles 115 from entering the skin when the multiple injection needles 115 have entered the skin to a predetermined depth.

Herein, the needle support 411 comprises, instead of the three columnar abutments 3121-3123 in the needle support 311 of the drug solution injection needle 310 according to Embodiment 3, a cylindrical abutment 4121 arranged at the center of the needle protruding surface 413 as the abutment section 412. Herein, the cylindrical abutment 4121 used as the abutment section 412 is arranged away from the injection needles 115 and arranged to avoid the space between adjacent injection needles 115 among the multiple injection needles 115, as shown in FIG. 8(b), such that an observable wheal is formed around each of the multiple injection needles 115.

For instance, the cylindrical abutment 4121 is provided with a space (e.g., 1 mm to 10 mm) sufficient for an observable wheal to form between the cylindrical abutment 4121 and the injection needles 115. The height of the cylindrical abutment 4121 is, for example, 1 mm to 10 mm, and the diameter of the cylindrical abutment 4121 is, for example, 1 mm to 5 mm. Further, a space corresponding to the height of the abutment section 412 (cylindrical abutment 4121), i.e., space from the needle protruding surface 413 to a skin abutment surface 412b of the cylindrical abutment 4121, is a wheal formation space 412a for ensuring that swelling of the skin by injection of a drug solution is not inhibited. Herein, the cross-sectional shape of the abutment section 412 is not limited to a circular shape. The cross-sectional shape may be oval or polygonal.

A needle support cap 411a, needle support main body 411b, outer shell section 401, needle guiding member 402, needle through hole 413a, flange 414, engagement bore 411a1, engagement protrusion 411b1, aperture 411a2, and rotation restricting protrusion 411b2 in the needle support 411 of the transdermal drug delivery device 400 according to Embodiment 4 are identical to the needle support cap 311a, needle support main body 311b, outer shell section 301, needle guiding member 302, needle through hole 313a, flange 314, engagement bore 311a1, engagement protrusion 311b1, aperture 311a2, and rotation restricting protrusion 311b2 in Embodiment 3, respectively.

Also in Embodiment 4, the plunger 123 is pushed in to inject a drug solution into subcutaneous tissue of a patient while the injection needle 115 is inserted into the skin until the cylindrical abutment 4121 of the transdermal drug delivery device 400 abuts the skin. While in such a state where the injection needle 115 is inserted into the skin, the positional relationship between a puncture site Rn punctured by the injection needle 115 and an abutment region Rc where the skin abutment surface 412b of the abutment section 412 abuts the skin surface is as shown in FIG. 8(d). That is, the abutment region Rc and the puncture site Rn are positioned with a space sufficient or more than sufficient for an observable wheal Wh to form therebetween. The abutment region Rc is not present between the puncture sites Rn of adjacent injection needles 115.

Embodiment 4 with such a configuration has the needle support 411 for supporting the multiple injection needles 115 such that the tip of each injection needle protrudes out from the drug solution injection needle 410 for transdermal administration of a drug solution. Thus, the locations of drug solution injection can be spread out.

Further, as the abutment section 412 for obstructing the multiple injection needles 115 from entering the skin by abutting the skin when the multiple injection needles 115 have entered the skin to a predetermine depth, the cylindrical abutment 4121 is provided with a space (e.g., 1 mm to 10 mmm) sufficient for an observable wheal to form between the cylindrical abutment 4121 and the injection needles 115. Thus, when the transdermal drug delivery device 400 according to Embodiment 4 is used to inject a drug solution into subcutaneous tissue, a wheal indicating that the drug solution has been injected can be observed.

Further, the cylindrical abutment 4121 is arranged at the center of the circular needle protruding surface 413 such that three injection needles 115 are positioned at a uniform interval around the cylindrical abutment 4121. Thus, the cylindrical abutment 4121 would not be present between adjacent injection needles 115, thereby preventing a wheal from being unlikely to form between adjacent injection needles 115 due to the cylindrical abutment 4121 for restricting the depth of puncture by an injection needle can prevent.

This results in embodiments of the present invention being able to realize the drug solution injection needle 410 that can not only inject a drug solution from multiple locations, but also restrict the depth of puncture by injection needles while preventing a wheal from being unlikely to form, and the transdermal drug delivery device 400 comprising the drug solution injection needle 410.

### Embodiment 5

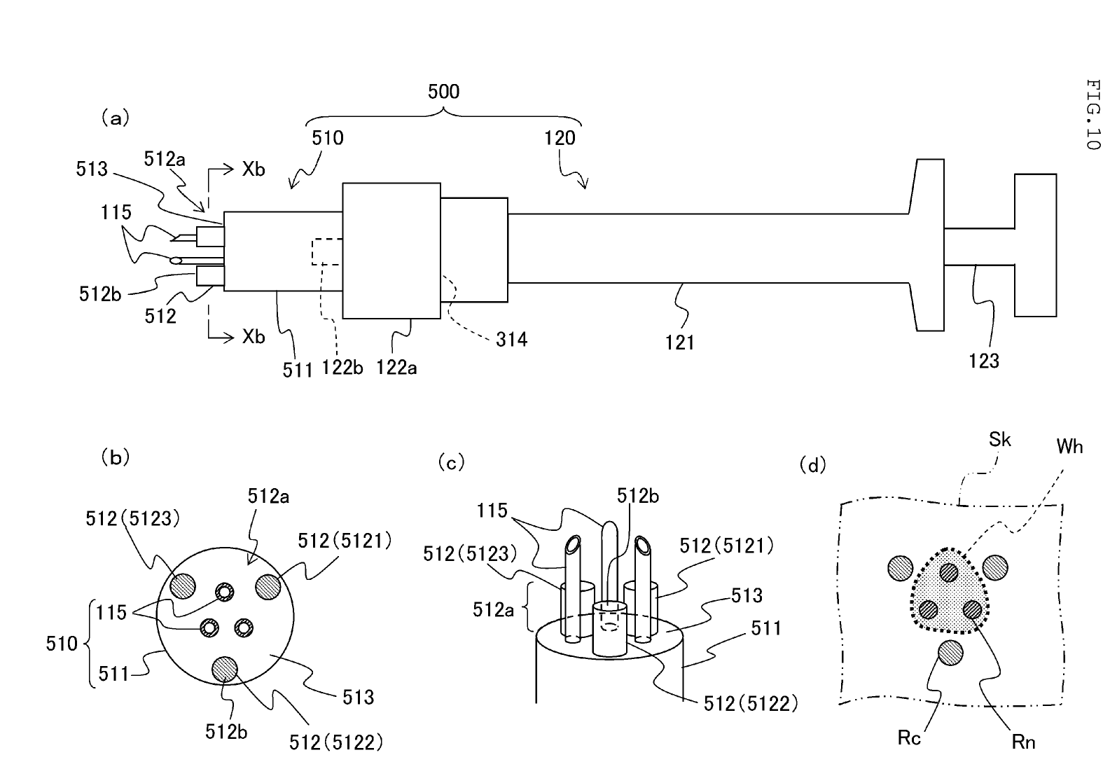

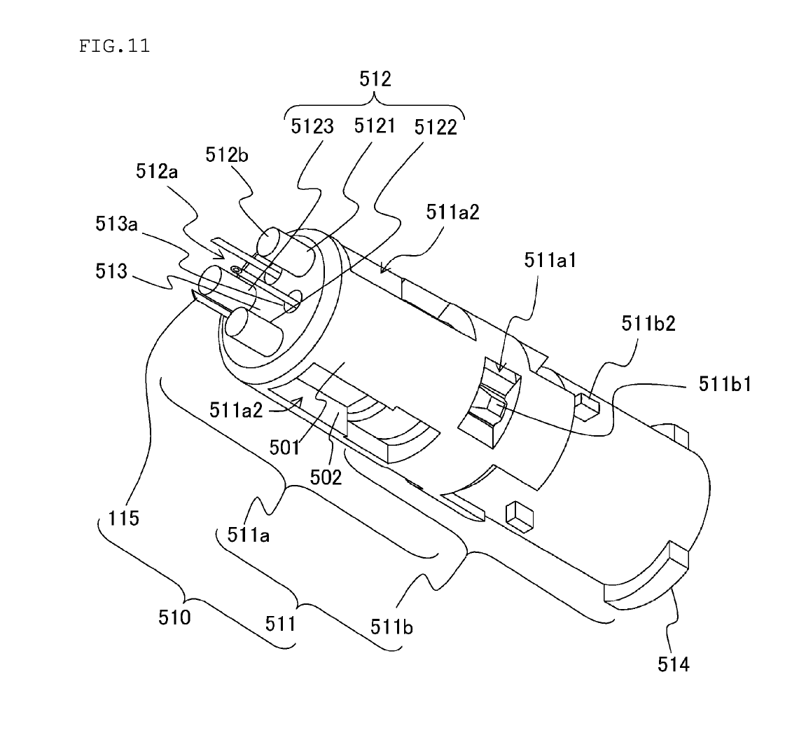

FIG. 10 a diagram for schematically explaining a transdermal drug delivery device according to Embodiment 5 of the present invention, showing the overall configuration of the transdermal drug delivery device (FIG. 10(a)), the structure of an injection needle portion (cross-section at the Xb-Xb line in FIG. 10(a)) of the transdermal drug delivery device (FIG. 10(b)), the structure of the tip of the injection needle portion of the transdermal drug delivery device (FIG. 10(c)), and the positional relationship of a puncture site punctured by the injection needle and an abutment region abutted by an abutment section on the skin (FIG. 10(d)). FIG. 11 is a perspective view showing the specific structure of the injection needle portion of the transdermal drug delivery device according to Embodiment 5 of the present invention.

A transdermal drug delivery device 500 according to Embodiment 5 comprises a drug solution injection needle 510 with a structure that is different from the drug solution injection needle 110 according to Embodiment 1, the drug solution injection needle 210 according to Embodiment 2, the drug solution injection needle 310 according to Embodiment 3, and the drug solution injection needle 410 in the transdermal drug delivery device 400 according to Embodiment 4.

Specifically, the drug solution injection needle 510 has, as in the drug solution injection needles 110, 210, 310, and 410 according to Embodiments 1, 2, 3, and 4, multiple (three) injection needles 115 and a needle support 511 for supporting the injection needles 115. The needle support 511 has a circular needle protruding surface 513 from which the multiple injection needles 115 protrude out and an abutment section 512 that abuts the skin to obstruct the multiple injection needles 115 from entering the skin when the multiple injection needles 115 have entered the skin to a predetermined depth.

Herein, the needle support 511 comprises, instead of one cylindrical abutment 412 in the needle support 411 of the drug solution injection needle 410 according to Embodiment 4, three cylindrical abutments 5121-5123 as the abutment section 512. The three cylindrical abutments 5121-5123 are arranged at equidistance along the circumferential edge of the circular needle protruding surface 513. Further, the three injection needles 115 are positioned inside an equilateral triangular region formed by connecting the positions of the three cylindrical abutments 5121-5123. Herein, the cylindrical abutments 5121-5123 used as the abutment section 512 are arranged away from the injection needles 115 and arranged to avoid the space between adjacent injection needles 115 among the multiple injection needles 115, as shown in FIG. 10(b), such that an observable wheal is formed around each of the multiple injection needles 115.

For instance, adjacent injection needles 115 and the cylindrical abutments 5121-5123 are arranged with a space (e.g., 1 mm to 10 mm) sufficient for an observable wheal to form therebetween. Herein, the height of the cylindrical abutments 5121-5123 is, for example, 1 mm to 10 mm, and the diameter of each of the cylindrical abutments 5121-5123 is, for example, 0.5 mm to 2.0 mm. Further, a space corresponding to the height of the abutment section 512, i.e., space from the needle protruding surface 513 to a skin abutment surface 512b of each of the cylindrical abutments 5121-5123 used as the abutment section 512, is a wheal formation space 512a for ensuring that swelling of the skin by injection of a drug solution is not inhibited. Herein, the cross-sectional shape of the columnar abutments 5121-5123 used as the abutment section 512 is not limited to a circular shape. The cross-sectional shape may be oval or polygonal.

A needle support cap 511a, needle support main body 511b, outer shell section 501, needle guiding member 502, needle through hole 513a, flange 514, engagement bore 511a1, engagement protrusion 511b1, aperture 511a2, and rotation restricting protrusion 511b2 in the needle support 511 of the transdermal drug delivery device 500 according to Embodiment 5 are identical to the needle support cap 411a, needle support main body 411b, outer shell section 401, needle guiding member 402, needle through hole 413a, flange 414, engagement bore 411a1, engagement protrusion 411b1, aperture 411a2, and rotation restricting protrusion 411b2 in Embodiment 4, respectively.

Also in Embodiment 5, the plunger 123 is pushed in to inject a drug solution into subcutaneous tissue of a patient while the injection needle 115 is inserted into the skin until the cylindrical abutments 5121-5123 in the needle support 511 of the drug solution injection needle 510 abuts the skin. While in such a state where the injection needle 115 is inserted into the skin, the positional relationship between a puncture site Rn punctured by the injection needle 115 and an abutment region Rc where the skin abutment surface 512b of the abutment section 512 abuts the skin surface is as shown in FIG. 10(d).

That is, the abutment region Rc and the puncture site Rn are positioned with a space sufficient or more than sufficient for an observable wheal Wh to form therebetween. The abutment region Rc is not present between the puncture sites Rn of adjacent injection needles 115.

Embodiment 5 with such a configuration has the needle support 511 for supporting the multiple injection needles 115 such that the tip of each injection needle protrudes out from the drug solution injection needle 510 for transdermal administration of a drug solution. Thus, the locations of drug solution injection can be spread out.

Further, as the abutment section 512 for obstructing the multiple injection needles 115 from entering the skin by abutting the skin when the multiple injection needles 115 have entered the skin to a predetermined depth, the cylindrical abutments 5121-5123 are provided with a space (e.g., 1 mm to 10 mmm) sufficient for an observable wheal to form between the cylindrical abutments 5121-5123 and the injection needles 115. Thus, when the transdermal drug delivery device 500 according to Embodiment 5 is used to inject a drug solution into subcutaneous tissue, a wheal indicating that the drug solution has been injected can be observed.

Further, the cylindrical abutments 5121-5123 are arranged to avoid the space between adjacent injection needles among the multiple injection needles 115. Thus, the cylindrical abutments 5121-5123 would not be present between adjacent injection needles, thereby preventing a wheal from being unlikely to form due to the cylindrical abutments 5121-5123 for restricting the depth of puncture by an injection needle.

This results in embodiments of the present invention being able to realize the drug solution injection needle 510 that can not only inject a drug solution from multiple locations, but also restrict the depth of puncture by injection needles while preventing a wheal from being unlikely to form, and the transdermal drug delivery device 500 comprising the drug solution injection needle 510.

The Embodiments show a specific structure of an abutment section. However, an abutment section is not limited to the above-described embodiments. Any abutment section may be used as long as it is provided away from an injection needle such that an observable wheal is formed around each of the multiple injection needles. In this case, it is desirable that the abutment section is provided to avoid the space between adjacent injection needles among the multiple injection needles.

As described above, aspects of the present invention are exemplified by the use of the example Embodiments. However, the present invention should not have an interpretation limited to such Embodiments. It is understood that the scope of the present invention should be interpreted solely based on the claims. It is also understood that those skilled in the art can implement an equivalent scope based on the descriptions of the present invention and common general knowledge from the descriptions of the specific preferred embodiments of the present invention. It is understood that the patent documents cited herein are incorporated herein by reference in the same manner as the contents that are specifically described herein.

The present invention is useful in the field of drug solution injection needles and transdermal drug delivery devices as a device capable of providing a drug solution injection needle that can not only inject a drug solution from multiple locations, but also restrict the depth of puncture by injection needles while preventing an observable wheal from being unlikely to form, and a transdermal drug delivery device comprising such a drug solution injection needle.

1-8. (canceled)

9. A drug solution injection needle having multiple injection needles and a needle support for supporting the multiple injection needles such that tip portions of the multiple injection needles protrude out,
wherein the needle support has
a needle protruding surface from which the multiple injection needles protrude out, and
an abutment section that abuts skin to obstruct the multiple injection needles from entering the skin when the multiple injection needles have entered the skin to a predetermined depth, the abutment section being provided on the needle protruding surface, and
wherein the abutment section is provided away from the injection needles such that an observable wheal is formed around each of the multiple injection needles.

10. The drug solution injection needle of claim 9, wherein the abutment section is provided to avoid a space between adjacent injection needles among the multiple injection needles.

11. The drug solution injection needle of claim 9, wherein the abutment section is arranged to surround the multiple injection needles along a circumferential edge of the needle protruding surface.

12. The drug solution injection needle of claim 10, wherein the abutment section is arranged to surround the multiple injection needles along a circumferential edge of the needle protruding surface.

13. The drug solution injection needle of claim 9, wherein the abutment section is discretely arranged at a predetermined interval along a circumferential edge of the needle protruding surface.

14. The drug solution injection needle of claim 10, wherein the abutment section is discretely arranged at a predetermined interval along a circumferential edge of the needle protruding surface.

15. The drug solution injection needle of claim 9, wherein the abutment section is arranged along a circumferential edge of the needle protruding surface so as to form a tubular shape.

16. The drug solution injection needle of claim 10, wherein the abutment section is arranged along a circumferential edge of the needle protruding surface so as to form a tubular shape.

17. The drug solution injection needle of claim 11, wherein the abutment section is arranged along a circumferential edge of the needle protruding surface so as to form a tubular shape.

18. The drug solution injection needle of claim 12, wherein the abutment section is arranged along a circumferential edge of the needle protruding surface so as to form a tubular shape.

19. The drug solution injection needle of claim 9, wherein the multiple injection needles are arranged to be positioned at vertices of a polygon inside the needle protruding surface, and
wherein the abutment section is arranged in a region inside the polygon.

20. The drug solution injection needle of claim 10, wherein the multiple injection needles are arranged to be positioned at vertices of a polygon inside the needle protruding surface, and
wherein the abutment section is arranged in a region inside the polygon.

21. A transdermal drug delivery device having a syringe filled with a drug solution and a drug solution injection needle attached to a tip section of the syringe,
wherein the drug solution injection needle comprises
multiple injection needles and
a needle support for supporting the multiple injection needles such that tip portions of the multiple injection needles protrude out,
wherein the needle support has
a needle protruding surface from which the multiple injection needles protrude out, and
an abutment section that abuts skin to obstruct the multiple injection needles from entering the skin when the multiple injection needles have entered the skin to a predetermined depth, the abutment section being provided on the needle protruding surface, and
wherein the abutment section is provided away from the injection needles such that an observable wheal is formed around each of the multiple injection needles.

22. The transdermal drug delivery device of claim 21, wherein the abutment section is provided to avoid a space between adjacent injection needles among the multiple injection needles.
